(19) United States
(12) Patent Application Publication
(10) Pub. No.: US 2022/0215912 A1
(43) Pub. Date: Jul. 7, 2022

(54) PATIENT MANAGEMENT SYSTEM

(71) Applicant: Drägerwerk AG & Co. KGaA, Lübeck (DE)

(72) Inventors: Götz KULLIK, Lübeck (DE); Frank FRANZ, Lübeck (DE); Birger LANDWEHR, Lübeck (DE)

(21) Appl. No.: 17/568,349

(22) Filed: Jan. 4, 2022

(30) Foreign Application Priority Data
Jan. 5, 2021 (DE) 10 2021 100 063.7

(51) Int. Cl. G16H 10/60; G16H 40/20

(52) U.S. Cl. CPC G16H 10/60; G16H 40/20

(57) ABSTRACT

A patient management system (100) manages an assignment to a patient, with a plurality of patient identifiers (110, 110′, 110″) and with a central patient monitoring device (120). Each of the patient identifiers has patient-specific information (112) stored thereon and is assigned to one of a plurality of patients. Each patient identifier includes a communication interface (114) configured to wirelessly output the patient-specific information. The central patient monitoring device is configured to detect and to store an assignment between the patient-specific information and the patient identifier based on the output (116) of the communication interface of the patient identifier. The patient monitoring device is configured to receive a forwarded signal (126) assigned to the patient identifier or to the patient-specific information and to process this forwarded signal based on the stored assignment.

### CROSS REFERENCE TO RELATED APPLICATIONS

This application claims the benefit of priority under 35 U.S.C. § 119 of German Application 10 2021 100 063.7, filed Jan. 5, 2021, the entire contents of which are incorporated herein by reference.

### TECHNICAL FIELD

The present invention pertains to a patient management system for managing an assignment to a patient. The present invention pertains, furthermore, to a process for managing an assignment to a patient.

### TECHNICAL BACKGROUND

It is common in a hospital setting to assign the responsibility for certain patients to a medical professional staff for the duration of a work shift. For example, a number of patients are assigned to each nursing staff member at the beginning of the service. In order to ensure that at least one medical professional, e.g., a nursing staff member, is assigned to each patient, the current assignment may be stored, for example, in a management system.

It is known from DE 102017010149 A1 for the transfer of the responsibility for a patient at the time of a change of shifts that a mobile device assigned to a first nursing staff member communicates for this purpose with a mobile device assigned to a second nursing staff member. The patient transfer is started by one of these devices and the second mobile device receives the assignment, the assignment information proper running in the background over a server, which stores this assignment information. Change of the assignment can be indicated, for example, by these two devices being brought together in space.

### SUMMARY

An object of the present invention is to provide an improved patient management system, and in particular an especially transparent and especially reliable patient management system.

A patient management system for management and assignment to a patient, with a plurality of patient identifiers and with at least one central patient monitoring device is provided according to the present invention for accomplishing this object.

A respective patient identifier from a plurality of patient identifiers, in which corresponding patient-specific information is stored for a respective patient from a plurality of patients, wherein the respective patient identifier further comprises a communication interface and wherein the respective patient identifier is configured to output the respective patient-specific information in a wireless manner (wirelessly) via the communication interface, especially to output the patient-specific information at recurring time intervals.

The at least one central patient monitoring device is configured to detect and to store an assignment between the patient-specific information and the patient identifier based on the output of the communication interface of the patient identifier. Furthermore, the patient monitoring device is configured to receive a forwarded signal assigned to the patient identifier or to the patient-specific information, especially to receive the forwarded signal from a medical device intended for monitoring the corresponding patient, and to process this forwarded signal on the basis of the assignment being stored.

It was found within the framework of the present invention that it cannot be detected in a clinical setting for a specific medical staff worker (nursing staff member) how many patients are assigned to this medical staff member. As such a current load of this medical staff member can not readily be taken into consideration in the distribution of acute tasks. In addition, it was found that the assignment of a patient and nursing staff or medical staff member must be carried out so reliably that it will always be recognized immediately when no nursing staff member who is currently present is assigned to a patient. The wearing of a patient identifier assigned to a specific patient by the nursing staff member is provided according to the present invention for solving this problem.

A nurse or nursing staff member or medical staff member is defined within the framework of the present invention as a person who helps a patient in recovery. He or she may be a nurse, hereinafter also called medical staff member, a physician or the like.

The patient management system according to the present invention makes it advantageously possible for the forwarded signal pertaining to a patient to be able to be outputted by the processing of the corresponding assignment by the patient monitoring device directly at a suitable location in order to trigger a response of the medical staff member having the patient identifier, which response is intended by the forwarded signal. The storage of the assignment between patient identifier and medical staff member in the central patient monitoring device may be carried out here according to the present invention at least partially manually or in an automated manner. As an alternative or in addition, an assignment between medical staff member and patient identifier may advantageously be eliminated, as a result of which a management effort within the framework of the patient management system according to the present invention can be reduced.

The patient management system can advantageously be adapted to an existing hospital processing system in a simple manner. Thus, only the plurality of patient identifiers, which are robust objects that are easy to prepare, and which make it possible according to the present invention to store the patient-specific information and have the communication interface, must be provided. The central patient monitoring device complements prior-art patient monitoring devices to the effect that the output of the communication interface of the patient identifier is detected and is used for the assignment of the patient-specific information to the patient identifier.

It can be reconstructed by the regular output of the patient-specific information via the communication interface whether the patient identifiers and hence the user of the patient identifier are in the vicinity of a corresponding receiving unit. In addition, in can be recognized from the repeated output whether the patient identifiers currently being used are operable.

The patient-specific information is a character string, which can be unambiguously assigned to the patient, e.g., a name, a registration number, a hospital bed number, an image or the like and/or a combination thereof. The character string must be suitable for inferring the patient currently assigned to this character string on the basis of the character string.

A respective patient identifier is according to the present invention a physical object which must be handed over physically at the time of a change in the responsibility for a specific patient, for example, at the time of a change of shifts. It is possible hereby to always ensure even in case of a great fluctuation of the responsibilities for a patient and/or of a great fluctuation of patients that no patient will remain without an assigned medical staff member. The patient identifier is preferably assigned to the corresponding patient and is given to each medical staff member preferably at the beginning of the treatment, for example, at the time of admission of the patient to the hospital, and the medical staff member who is in possession of this patient identifier will know from then on that he or she is responsible for this patient. The assignment to the medical staff member can therefore be carried out by the patient identifier as a physical representation of the assignment, without this assignment having to be stored digitally with a corresponding management effort.

A plurality of patient identifiers may be assigned according to the present invention to a single patient. This may happen, for example, when both a nursing staff member or medical staff member and a physician each wear a corresponding patient identifier each for this patient. For example, it can be ensured hereby following a surgery that an attending physician will also be informed of the current state of the patient, in addition to a nursing staff member. In addition, a patient identifier assigned to the patient in question may also be handed out, for example, to a visitor, in addition to one handed out to a nursing staff member. Information on the user or at least on a status or on a role of the user of the patient identifier is preferably stored on the corresponding patient identifier.

The plurality of patient identifiers may be structured such that a nursing staff member can wear all these patient identifiers assigned to this nursing staff member comfortably and in such a manner that they are visible from the outside. A number of patient identifiers may have for this purpose at least partially such a connection structure that a plurality of patient identifiers can be attached to one another, and especially such that they can be pinned onto one another. Such patient identifiers pinned to one another may be worn, for example, as a clip, as a bracelet, as a necklace or the like. As an alternative or in addition, at least one mobile user carrier device of the patient management system is provided, on which a number of patient identifiers can be arranged, especially such that they can be detected optically (viewed or visually recognized) from the outside. Such a user device may be carried, for example, as a clip, as a bracelet, as a necklace or the like.

The forwarded signal may pertain, for example, to alarm generation information and/or to treatment information. The alarm generation information and/or the treatment information are preferably assigned to a patient via the patient-specific information.

Preferred embodiments of the patient management system according to the present invention will be described below.

At least one patient identifier from the plurality of patient identifiers preferably comprises a sensor unit, which is configured to detect a motionless state of the patient identifier. The sensor unit is preferably an acceleration sensor and/or a location sensor. The detection of a continual motionlessness preferably triggers now the output of an alarm. It can advantageously be avoided hereby that a patient identifier is accidentally forgotten, lost and/or put down.

In one embodiment, in which a mobile user carrier device is provided for a respective nursing staff member in order to store a number of patient identifiers assigned to this nursing staff member on the mobile user carrier device, the mobile user carrier device is configured to store at most 10 patient identifiers, especially at most 8 patient identifiers, and especially preferably at most 6 patient identifiers in an optically detectable manner.

A corresponding patient identifier from the plurality of patient identifiers preferably comprises, furthermore, an output unit and is configured to trigger a corresponding output, especially an acoustic, optical and/or tactile output, by the output unit after receiving a corresponding signal.

In an especially preferred embodiment, a respective patient identifier has, furthermore, an energy storage device. The output of the patient-specific information can preferably be carried out via the communication interface by means of the energy provided by the energy storage device. The energy storage device is preferably a replaceable battery. As an alternative or in addition, the energy storage device is a rechargeable battery which can be charged in a contactless manner. In an alternative embodiment, the output is carried out via the communication interface without additionally provided energy, for example, by a reading mechanism, for example, via a passively contacted Radio Frequency Identification (RFID) tag.

In a preferred embodiment, a number of patient identifiers from the plurality of patient identifiers have an internal memory, which is configured to store the patient-specific information at least for a duration of treatment of the corresponding patient. This memory is preferably loaded with the patient-specific information at the beginning of the treatment of a patient, for example, at the time of admission into a hospital. As an alternative, the patient identifier may always have the same patient-specific information, which is assigned to the patient at the beginning of the treatment of a patient and it is as a result patient-specific information for this patient. Consequently, patients who are not treated simultaneously in the hospital but to whom the same patient identifier has been assigned also have the same patient-specific information, namely, the information assigned to the patient identifier.

In a preferred embodiment, the patient monitoring device is configured to output a patient signal assigned to the respective patient identifier to the respective patient identifier, and the respective patient identifier is further configured to receive the patient signal, preferably to receive it via the communication interface, and to trigger a corresponding output. The output may be carried out here directly at the respective patient identifier via an output unit, such as a display and/or a speaker or the like. As an alternative or in addition, the output may be outputted at a separate external device, to which the patient identifier is communicated via a corresponding interface, e.g., likewise via the communication interface. In a variant of this embodiment, the patient identifier has at least two interfaces for communication with other devices, namely, at least the communication interface for the communication with the patient monitoring device and another interface for the communication with an external device, especially with an adjacent external device. In an especially preferred variant of the above embodiment, the output is carried out as a tactile output, optical output and/or acoustic output. A tactile output may be, for example, a vibration of a mobile device being carried out or a haptic feedback of an adjusting knob.

In an especially preferred variant of the above exemplary embodiment, the corresponding output of the corresponding patient identifier comprises a wireless output of an alarm generation signal to an external output device, which is located in a predefined surrounding space, in order to trigger an alarm generation output or a treatment output by the external output device. The patient identifier makes possible especially advantageously in this variant a purposeful device output in the vicinity of the user of the patient identifier and hence, via the central patient monitoring device, a reliable notification of the relevant medical staff member, if information concerning an alarm generation and/or a treatment shall be outputted concerning a patient currently being treated. As a result, an especially rapid and reliable notification is possible. The external output device may be, for example, a floor display, a medical device, a computer, an alarm generation device in the break room of the medical staff or the like.

In an especially advantageous embodiment, the patient management system has, furthermore, at least two locating units, via which a position of at least one patient identifier in a region (building or campus) can be determined, wherein the patient monitoring device is configured to assign the correspondingly determined position in the region (building or campus) to the patient identifier or to the patient-specific information. A current location of the patient identifier can especially advantageously be assigned in this embodiment to each patient via the assignment to the patient identifier. It is possible as a result to decide, for example, in case of an acute alarm, whether the assigned patient identifier is informed or whether a patient identifier located markedly closer to the patient is informed about the patient to be treated. Furthermore, it can be determined by the use of the locating units whether the patient identifier currently assigned to the patient is in the environment of the hospital at all and it may possibly also be determined when it has left this environment. For example, it can be detected in this manner whether the assigned medical staff member is having a break right now, for example, a smoking break, outside the hospital (outside of a particular region). The determination of a position by two locating units is carried out, for example, by a measurement of a signal intensity of an output of a respective patient identifier by both locating units, so that a location within the hospital can be determined on the basis of a distance to both locating units. As an alternative or in addition, a location can be determined by means of a third locating units by triangulation of the corresponding signals in the known manner. The use of such locating units is known to the person skilled in the art and it will not therefore be explained in more detail below.

In a variant of the above embodiment, the patient monitoring unit is configured to trigger a device output, especially an alarm generation output, or a treatment output, by the external output device depending on the assigned position at an external output device located in the space surrounding this assigned position. The fact that the position of the corresponding patient identifier is known by means of the at least two locating units is utilized in this variant.

An external output device in the area surrounding this position can thus be actuated directly to output information relevant for the user of the patient identifier, especially information concerning the state of the assigned patient, in a detectable manner.

The patient monitoring device is preferably connected to an alarm system of the corresponding institution, for example, of the hospital, so that an alarm generation triggered by the alarm system can be displayed via the patient management system according to the present invention to the user of the patient identifier, especially via an external output device and/or via the patient identifier.

In a preferred embodiment, the patient monitoring device is further configured to trigger a disconnection output if a communication interface with a monitored patient identifier from the plurality of patient identifiers is disconnected. It is advantageously ensured in this embodiment that in case of a technical defect or if the area being monitored is left, a disconnection output takes place in order to provide information about the fact that the medical staff member assigned to a patient is not currently probably available for the care of the patient. The patient monitoring device is preferably configured, furthermore, in this connection to wait over a predefined time interval before an actual, perceptible output takes place after the disconnection output has been triggered. It is ensured thereby that a temporary disconnection problem or the fact that the area being monitored has been left for a short time does not lead to a disconnection output and does not lead to a corresponding management effort.

In one embodiment of the patient management system according to the present invention, role information, which pertains to an intended role to be played by the user of the corresponding patient identifier from a predefined group of roles, is assigned to a respective patient identifier in the patient monitoring device, wherein this predefined group comprises at least one of the following roles: Nursing staff member, physician, visitor, trainee, temporary employee. The role information makes possible an assignment of authorization levels to the respective patient identifier, depending on the existing role. Thus, the communication between the respective patient identifier and an external device may depend on the role assigned to the patient identifier. For example, certain patient-specific information, for example, information that is subject to data protection, are not outputted to a trainee. Furthermore, the communication between patient identifier and an external medical device may release an operation of the medical device. For example, a physician may be allowed to adjust a parameter of an external medical device, whereas the visitor, the trainee and/or the temporary employee cannot perform such an adjustment of the parameter.

In an especially preferred embodiment, the forwarded signal is processed corresponding to a predefined forwarding instruction (forwarding rule). This forwarding instruction assigns to a group of possible forwarded signals a mode of processing assigned to this group. For example, an important alarm can be outputted to other patient identifiers as a notification that is not time-critical.

In an especially preferred variant of the above two embodiments, the predefined forwarding instruction pertains to a forwarding to a selection of a subset of roles from the predefined group of roles. The forwarded signal is forwarded in this embodiment depending on the role information that is assigned to the patient identifier pertaining to the patient assigned to the forwarded signal. Should there be a plurality of patient identifiers for the corresponding patient-specific information, the forwarded signal is processed corresponding to the respective role of the patient identifier. For example, a visitor will not be assigned a forwarded signal at his patient identifier if this signal indicates treatment information. An especially important alarm is preferably sent to a patient identifier to which the role of a physician is assigned. Aspects of data protection and the goal of having an especially small number of acoustic alarms in the hospital environment can especially advantageously be taken into consideration by taking into consideration the stored role information for a patient identifier.

At least one respective patient identifier from the plurality of patient identifiers preferably has a plastic cover or a silicone cover, particularly a silicone cover. Cleaning and disinfection of the patient identifier is possible in an especially simple manner due to the provision of such a cover. This is especially advantageous because the patient identifier is changed physically over from one medical staff member to the next at the time of a change of shifts at the latest, so that regular cleaning of this cover is necessary. The provision of a cover can especially advantageously lead to a smooth surface of the patient identifier, and such a surface can be cleaned and/or disinfected in an especially simple manner.

A respective patient identifier is configured in another embodiment such that it makes possible an unambiguous optical identification (provides unambiguous optical identifiability) of the patient assigned to the patient identifier based on a color, a symbol, a character string, indicia, a mark or the like. Not only can the number of the patient identifiers currently assigned to the medical staff member be detected optically in this embodiment, but it is also possible to detect a specific patient or at least one patient classification of the corresponding patient, e.g., a patient in the intensive care unit, a comatose patient, or a patient in a postoperative state. It is, for example, possible as a result to optically detect how much care is expected to be needed for a patient assigned to the medical staff member corresponding to the patient identifier that is carried along. The assignment of a color within the framework of this embodiment may correspond, for example, to a bed color assigned to the patient, to a door color of the hospital room or the like.

According to another aspect of the present invention, a process for managing an assignment to a patient is proposed to accomplish the abovementioned object, said process having the following steps:
assignment of a plurality of patients to a patient identifier each and storage of corresponding patient-specific information;
wireless outputting of the patient-specific information by the patient identifier;
detection and storage of an assignment between the patient-specific information and the patient identifier based on the output;
receiving of a forwarded signal assigned to the patient identifier or to the patient-specific information; and
processing of the forwarded signal based on the stored assignment.

The process according to the present invention is carried out by the patient management system according to the present invention and it thus has all the advantages described for this system. In particular, the process according to the present invention makes possible an especially reliable assignment between patient and medical staff, because this assignment is not carried out at the abstract level or it is carried out not only at the abstract level, for example, by a work instruction and/or by the storage of this assignment in a data bank, but at the physical level by the possession of the patient identifier for a specific patient by a medical staff member. Especially at the time of a change of shifts, the transfer of a responsibility for a patient can be carried out in an especially simple manner, rapidly and reliably. In addition, the responsibility for a patient can also be transferred for a short time in a simple manner, rapidly and reliably in order to make it possible for the medical staff, for example, to temporarily leave a monitored area, e.g., a hospital ward. It may be necessary to temporarily leave the monitored area, for example, because of a task of transporting a patient, an emergency in another ward or based on a temporary break.

The carrying out of the process described is typically preceded by a provision of the plurality of patient identifiers and of the at least one mobile user carrier device as a preceding step.

The process according to the present invention may also be expanded by one or more process steps corresponding to the advantageous embodiments described for the patient management system.

The additional steps of receiving a patient signal assigned to the patient identifier and of triggering a corresponding output in response to the patient signal are carried out in an advantageous embodiment.

In an alternative or additional embodiment, the process according to the present invention has, furthermore, a detection and storage of an assignment between the patient-specific information and the user device based on the output of the user device.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of a patient management system according to the present invention;

FIG. 2 is a schematic view of a second exemplary embodiment of the patient management system according to the present invention;

FIG. 3 is a schematic view of a third exemplary embodiment of the patient management system according to the present invention; and

FIG. 4 is a flow chart of an exemplary embodiment of a process according to another aspect of the present invention.

### DESCRIPTION OF PREFERRED EMBODIMENTS

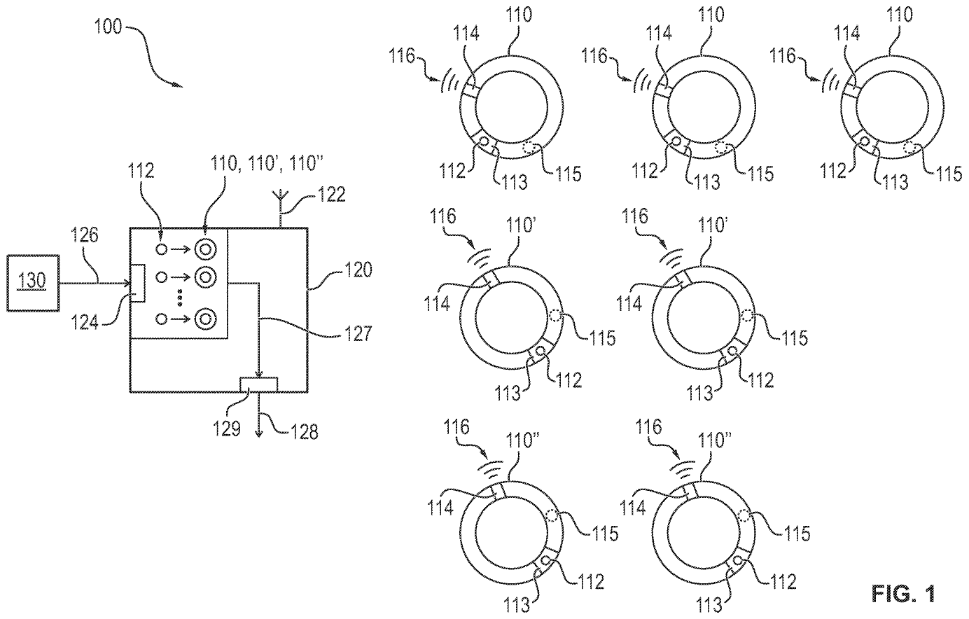

Referring to the drawings, FIG. 1 shows a schematic view of a first exemplary embodiment of a patient management system 100 according to the present invention.

The patient management system 100 is configured to manage an assignment to a patient. The patient management system 100 has for this purpose a plurality of patient identifiers 110, 110′, 110″ and at least one central patient monitoring device 120.

Each patient identifier 110, 110′, 110″, in which corresponding patient-specific information 112 is stored, is assigned to a corresponding one of a plurality of patients. In the exemplary embodiment shown, the three patient identifiers 110 are assigned to a nursing staff member or medical staff member, namely, to a nurse or other medical staff member, and the two patient identifiers 110′ are assigned to another medical staff member, namely, to a physician, and the two patient identifiers 110″ are assigned to another medical staff member, namely, to another nurse. The patient identifiers 110, 110′ and 110″ are configured in the form of a bracelet, so that a plurality of patient identifiers can be worn simultaneously by the same person and the number of identifiers can also be detected optically by other persons, for example, other colleagues. Other possible forms of the patient identifier are shown within the framework of the other embodiments shown. Each patient identifier 110, 110′, 110″ comprises, in addition, a communication interface 114, via which the respective patient-specific information 112 is outputted in a wireless manner as an output signal 116, especially at recurring time intervals. The wireless communication may take place according to the present invention via WLAN, Bluetooth, BLE, ZigBee, LoRa or the like. Details on the structure of such an interface for the wireless communication are known to the person skilled in the art and will not therefore be explained below.

For storing the patient-specific information 112, each patient identifier 110, 110′, 110″ shown in the exemplary embodiment has an internal memory 113. The internal memory 113 is preferably configured to store the patient-specific information 112 at least for the duration of the treatment of the corresponding patient. New patient-specific information 112 is preferably stored in the patient identifier in case of the readmission of a patient. The patient-specific information 112 is preferably information characterizing the patient, for example, the patient's name, the patient's date of birth, the patient's health insurance number or the like. As an alternative or in addition, a character string, a symbol or the like may be permanently assigned as at least a portion of the patient-specific information, and after this patient has been discharged, a patient admitted later will have repeatedly the same patient-specific information assigned to that later admitted patient as at least a portion of the information individualizing the patient in the hospital.

Finally, the communication interface 114 is supplied with energy by a respective energy storage device 115 of the corresponding patient identifier 110, 110′, 110′. The energy storage device 115 may be, for example, a replaceable batter or a rechargeable battery, which can be charged in a contactless manner.

The at least one central patient monitoring device 120 of the patient management system 100 is configured to detect and to store an assignment between the patient-specific information 112 and the patient identifier 110, 110′, 110″ based on the output signal 116 of the communication interface 114. The patient monitoring device 120 has for this purpose an antenna unit 122 for receiving the corresponding output signal 116. Furthermore, the patient monitoring device 120 has a receiving interface 124 for receiving a forwarded signal 126 assigned to the patient identifier or to the patient-specific information. In an alternative or additional exemplary embodiment, the receiving interface and the antenna unit form a common interface for receiving different signals. The forwarded signal 126 is preferably a signal that is received from an external medical device 130 intended for monitoring the corresponding patient. As an alternative or in addition, the forwarded signal may also be provided by an another external device relevant for the treatment of the patient and/or for the processing of patient data.

The processing of the forwarded signal 126 is based here on the stored assignment between patient-specific information 112 and the corresponding patient identifier 110, 110′, 110″. As a result, the forwarded signal 126 is converted into a processed forwarded signal 127.

After the processing of the forwarded signal 126, a corresponding patient signal 128 is outputted via a wireless output interface 129 of the central patient monitoring device 120 in the exemplary embodiment shown.

The patient signal 128 can be outputted specifically to a respective patient identifier 110, 110′, 110″, to another external device, not shown, to another component of the patient management system or the like, as this is described as an example in the following exemplary embodiment.

At least one respective patient identifier 110, 110′, 110″ from the shown plurality of patient identifiers 110, 110′, 110″ preferably has a plastic cover, especially a silicone cover.

In one exemplary embodiment, not shown, a character string, a symbol, a color and/or an image can be recognized on a respective patient identifier, as a result of which the specific patient, who is assigned to this patient identifier, can be intuitively recognized. For example, each hospital bed in an intensive care unit may have a color of its own and the patient correspondingly assigned to this hospital bed receives a patient identifier assigned to that patient, which has the color of the bed. As a result, it would be possible for an outsider to intuitively assign visually the patient identifier currently being carried to the patient on the basis of the patient identifier.

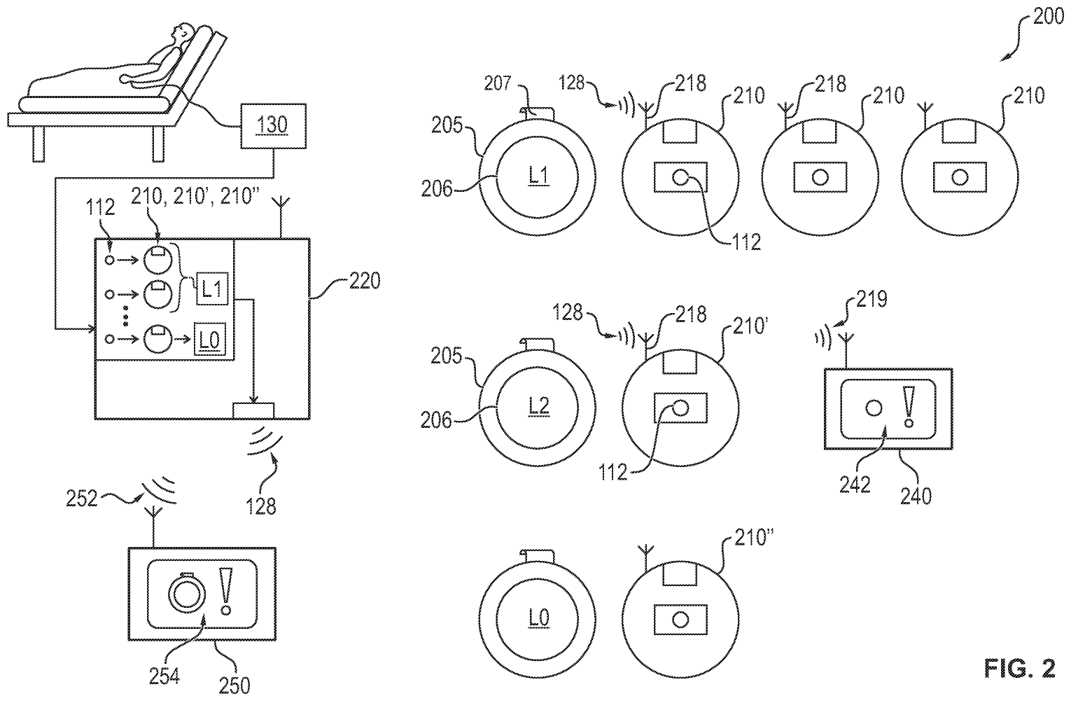

FIG. 2 shows a schematic view of a second exemplary embodiment of the patient management system 200 according to the present invention.

The patient management system 200 differs from the patient management system 100 shown in FIG. 1 in that the patient identifiers 210, 210′, 210″ have an antenna module 218 each, via which they can receive and process further the patient signal 128 provided by the central monitoring device 220. In the exemplary embodiment shown, an output to two patient identifiers 210, 210′, to both of which patient-specific information 112 is assigned by the patient signal 128, is triggered by the receipt of a corresponding patient signal 128. The first patient identifier 210 is assigned in this case to a medical staff member, such as a nurse, and the second patient identifier 210′ is assigned to an attending physician. The patient signal 128 is sent in this case, for example, to both patient identifiers, because it indicates an alarm, which both the attending physician and the responsible medical staff member shall perceive. The patient signal 128 is based here on a forwarded signal 126 from an external medical device 130, which is used for the treatment of the patient to whom the corresponding patient-specific information 112 is assigned as well. The patient identifier 210′ ensures in this case an optical display of the corresponding alarm indicated by the patient signal 128 on the floor display 240 via a corresponding communication with a floor display 240 located in the surrounding area by the output 219. As a result, the attending physician can immediately recognize in the physician's environment based on the corresponding optical output 242 that an alarm is present for the patient being treated by the physician. The patient identifier 210 of the medical staff member triggers an acoustic and tactile output directly at the patient identifier 210 based on the patient signal 128. The medical staff member can immediately recognize based on the vibrating patient identifier 210, which generates an alarm thereby, for which patient an alarm is being generated. The type of the existing alarm can preferably be inferred on the basis of the manner of the acoustic and tactile output. It is illustrated in the exemplary embodiment that different outputs based on the received patient signal 126 can be triggered by the respective patient identifier. The outputs described are only examples of other possibilities of an alarm generation, which is as direct as possible, for the person currently wearing the patient identifier 210, 210′.

Furthermore, the patient management system 200 shown has a communication connection between the central patient monitoring device 220 and an external management output device 250. As soon as a communication interface with a monitored patient identifier 210, 210′, 210″ from the plurality of patient identifiers is disconnected, a disconnection output 252, which is received by the management output devices, is triggered by the central patient monitoring device 220. It is displayed thereby at a central location, for example, in a central hospital management system, via an optical output 254 when the user of a patient identifier or of a number of patient identifier is not on the premises for monitoring the patients assigned corresponding to the patient-specific information. The disconnection output 252 is preferably triggered by the patient monitoring device 220 only if the communication interface with the corresponding patient identifier 210, 210′, 210″ is disconnected over a predefined time period, especially for more than 30 sec, especially preferably for more than 2 minutes, for example, for more than 5 minutes. It is ensured hereby that a temporary connection problem, a smoking break or the like does not lead to a corresponding disconnection output 252 and hence to a corresponding management effort.

Unlike the bracelet-shaped patient identifiers from FIG. 1, the patient identifiers 210, 210′, 210″ of the patient management system 200 are disk-shaped. A corresponding person, e.g., a medical staff member, has in this case a mobile user carrier device 205, which makes it possible to arrange a corresponding patient identifier 210 at the user device 205 by means of a corresponding connection structure. The connection structure 206 is extended in this case in the corresponding housing structure, not shown, of the patient identifiers, so that a plurality of patient identifiers can be attached to one another, especially pinned to one another, in order to be worn together at the mobile user carrier device 205. The user carrier device 205 has a clip 207, via which all the patient identifiers 210, 210′, 210″ assigned to the corresponding person can be attached together to the clothing of the corresponding person.

In one exemplary embodiment, not shown, the mobile user carrier device 205 is configured to communicate with at least one assigned patient identifier. Role information L0, L1, L2 is preferably assigned here to a person carrying the user device 205 and is passed on to the attached patient identifier. This makes it possible to detect an assignment between patient identifier and role information in an especially simple manner. The role information pertains in the exemplary embodiment shown to an intended role of the user of the corresponding patient identifier from a predefined group of roles, wherein this predefined group comprises at least one of the following roles: Nursing staff member, physician, visitor, trainee, temporary employee.

This role information is shown in the exemplary embodiment shown on the corresponding mobile user carrier device 205 for reasons of clarity.

Finally, the patient management system 200 differs from the patient management system 100 in that the role information concerning the user of the respective patient identifier is assigned to the respective patient identifier in the patient monitoring device 220. The forwarded signal 126 is processed in this case further depending on the role information L0, L1, L2 assigned to the patient-specific information 112. Thus, the forwarding in the form of the patient signal 128 is carried out only to a selection of a subset of roles from the predefined group of roles. For example, treatment information is not outputted to a visitor having the role LO. Furthermore, alarm information as was described above is outputted to the physician having the role L2 differently than to the medical staff member having the role L1. Hence, depending on the role assigned correspondingly, there are different forwarding instructions, which are stored in the central patient monitoring device 220. The role information may be present, for example, as indicated in the output of the patient identifiers and it can as a result be outputted especially advantageously in an automated manner. For example, the role information can be outputted via the user device to the assigned patient identifiers, for example, by means of passive or active RFID tag, in order to make possible a corresponding output with role information to the central patient monitoring device.

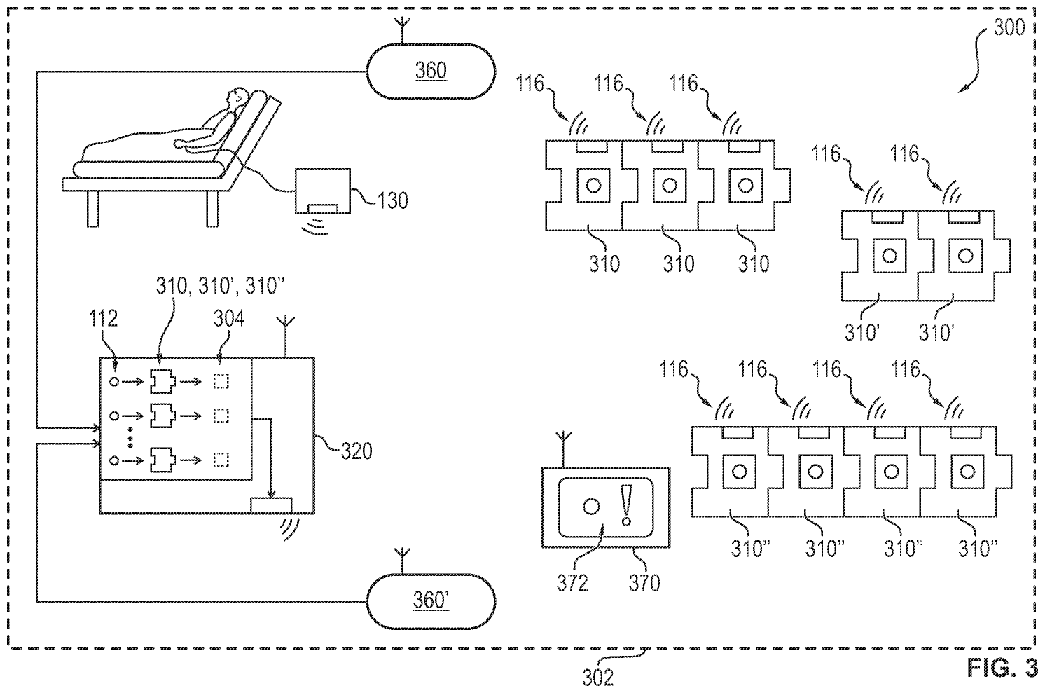

FIG. 3 shows a schematic view of a third exemplary embodiment of the patient management system 300 according to the present invention.

The patient management system 300 differs from the patient management system 200 shown in FIG. 2 in that the patient management system 300 comprises at least two locating units 360, 360′. The locating units 360, 360′ are configured to analyze a signal intensity of the corresponding output signals 116 of the respective patient identifiers 310, 310′, 310″ and thereby to determine a position 304 of at least one patient identifier 310, 310′, 310″ in the corresponding a region (building or campus) 302 and to output it to the central patient monitoring device 320. In an alternative or additional exemplary embodiment, the patient management system has at least one additional locating unit and is configured to determine the position of the at least one patient identifier in the region (building or campus), for example, by means of triangulation of the received signals.

The patient monitoring device 320 is further configured to assign the correspondingly determined position 304 in the region (building or campus) 302 to the patient identifier 310, 310′, 310″ or to the patient-specific information 112.

It is made possible by the assignment of the position 304 that a signal, which is to be correspondingly outputted, is outputted depending on the assigned position 304 after receipt of a forwarded signal 126, for example, from an external medical device 130. A device output 372, especially an alarm generation output or a treatment output, can thus be triggered in an area surrounding this assigned position 304 by a corresponding external output device 370. The user of the patient identifier is advantageously informed thereby of the device output 372, without a connection to the patient identifier having to be established before. An especially direct and simple communication with the person to be informed is advantageously possible thereby. In addition, the person to be informed does not have to carry along any large devices with a large display or the like, because the simple and small patient identifiers already make the communication possible via the external output device 370.

In the exemplary embodiment shown, the patient identifiers 310, 310′, 310″ have an outer structure that makes it possible for a plurality of patient identifiers to be able to be attached to one another. Such stacks of patient identifiers attached to one another may be attached, for example, via a necklace, a bracelet, a clip or the like to a piece of clothing of the user of the patient identifiers.

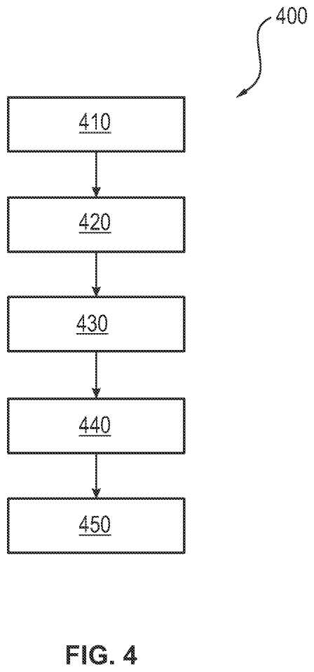

FIG. 4 shows a flow chart of an exemplary embodiment of the process 400 according to the present invention.

The process 400 according to the present invention is configured for managing an assignment to a patient. It has for this purpose the steps described below.

A first step 410 comprises an assignment of a plurality of patients to a patient identifier each and storage of corresponding patient-specific information.

A next step 420 comprises a wireless output of the patient-specific information by the patient identifier.

A next step 430 comprises the detection and storage of an assignment between the patient-specific information and the patient identifier based on the output.

A further step 440 comprises the receipt of a forwarded signal assigned to the patient identifier or to the patient-specific information.

A final step 450 comprises the processing of the forwarded signal based on the stored assignment.

Steps 410, 420, 430, 440 and 450 are preferably carried out in the order shown. The storage of the patient-specific information on the patient identifier can be carried out at different times relative to the further process steps. Thus, the patient-specific information may be stored permanently on the patient identifier, for example, by a bar code, a QR code (quick response code), an RFID tag or the like. The patient-specific information may be a fixed character string, which characterizes, for example, a specific bed location or the specific patient identifier. The patient-specific information is patient-specific in this case to the extent that this specific information, such as, e.g., the specific bed location or the specific patient identification, is unambiguously assigned to the patient at the beginning of his treatment, so that this information is stored specifically for this patient and thus it allows an unambiguous assignment between the patient-specific information and the patient. As an alternative or in addition, step 410 may take place at the beginning of the treatment of a patient, for example, at the time of his admission into the hospital. The patient-specific information is, for example, a name, a date of birth, the name of a health insurance number, or other characterizing information of the patient.

Step 420 may be carried out several times, especially at recurring time intervals, after step 410 has been carried out once, in order to ensure a communication connection between the patient identifier and the central patient monitoring device.

Step 430 is carried out typically immediately after step 420, because the assignment between the patient-specific information and the patient identifier is detected and stored directly after the output of the patient-specific information by the patient identifier. This storage may be carried out at least partly manually. As an alternative, patient-specific information and identification information of the patient identifier may be outputted together via the output of the patient identifier and thereby make possible an automated assignment and storage of this assignment.

Step 440 may take place at any desired time relative to the further process steps. Thus, the forwarded signal may also be received before the detection and storage of the assignment according to step 430. Steps 410, 420 and 430 are, however, preferably carried out before the processing of signals corresponding to this assignment. Thus, steps 440 and 450 are carried out typically sometime after the further process steps. These two steps are triggered by the forwarded signal, which is typically an alarm generation signal and/or a treatment signal, so that it is always uncertain for a specific patient when or whether such a signal must be received and processed at all in the course of the treatment of the patient.

The wireless output of the patient-specific information corresponding to step 420 of the process according to the present invention takes place typically at recurring time intervals, e.g., after a respective time interval of less than 10 minutes, preferably less than 5 minutes, and especially preferably less than 2 minutes.

The process 400 may be complemented according to the present invention by advantageous exemplary embodiments, for example, by the addition of one or more additional process steps. The process may thus comprise, in addition, for example, the step of receiving a patient signal and of a subsequent triggering of an output of the corresponding patient identifier. The output may be here, for example, an alarm generation output and/or a treatment output.

As an alternative or in addition, the process according to the present invention may additionally also comprise the process step of detecting and storing an assignment between the patient-specific information and the patient identifier based on the output of the patient identifier.

The communication between the patient identifier and the central patient monitoring device and between the central patient monitoring device and other possible devices preferably takes place in a wireless manner, because the patient identifier shall be free from cables to external devices.

While specific embodiments of the invention have been shown and described in detail to illustrate the application of the principles of the invention, it will be understood that the invention may be embodied otherwise without departing from such principles.

### LIST OF REFERENCE NUMBERS

100, 200, 300 Patient management system
110, 110′, 110″, 210, 210′ Patient identifier
210″, 310, 310, 310″
112 Patient-specific information
113 Internal memory
114 Communication interface
115 Energy storage device
116 Output signal
120, 220, 320 Central patient monitoring device
122 Antenna unit
124 Receiving interface
126 Forwarded signal
127 Processed forwarded signal
128 Patient signal
130 External medical device
205 Mobile user carrier device
206 Connection structure
207 Clip
218 Antenna module
219 Output of the patient identifier
240 Floor display
242, 254 Optical output
250 Management output device
252 Disconnection output
302 Region (building or campus
304 Position
360, 360′ Locating unit
370 External output device
372 Device output
400 Process
410, 420, 430, 440, 450 Process steps

What is claimed is:

1. A patient management system for managing an assignment to a patient, the patient management system comprising:
a plurality of patient identifiers, each of the respective patient identifiers comprising a communication interface, wherein each of the respective patient identifiers is configured to store patient-specific information of one of a corresponding plurality of patients and to output the patient-specific information wirelessly via the communication interface; and
a patient monitoring device configured to detect and to store an assignment between the patient-specific information and the patient identifier based on the output of the communication interface of the patient identifier, to receive a forwarded signal assigned to the patient identifier or to the patient-specific information and to process the forwarded signal based on the stored assignment.

2. A patient management system in accordance with claim 1, wherein each of the patient identifiers further comprises an energy storage device.

3. A patient management system in accordance with claim 1, wherein each of the patient identifiers comprises an internal memory configured to store the patient-specific information for a time period at corresponding to a duration of a patient treatment of the corresponding patient.

4. A patient management system in accordance with claim 1, wherein:
the patient monitoring device is configured to output a patient signal assigned to the respective patient identifier; and
each of the patient identifiers is further configured to receive the patient signal and to trigger a corresponding output.

5. A patient management system in accordance with claim 4, wherein the patient signal assigned to the respective patient identifier is outputted as at least one of a tactile output, an optical output and an acoustic output.

6. A patient management system in accordance with claim 4, wherein the output of the patient signal assigned to the respective patient identifier comprises a wireless output of an alarm generation signal to an external output device located in a predefined surrounding area to trigger an output by the external output device.

7. A patient management system in accordance with claim 1, further comprising two locating units, via which a position of at least one patient identifier in a region is determined, wherein the patient monitoring device is configured to assign the correspondingly determined position in the region to the patient identifier or to the patient-specific information.

8. A patient management system in accordance with claim 7, wherein:
the patient monitoring device is configured to trigger a device output by the external output device depending on the assigned position; and
the external output device is responsive based on a location of the external output device in a surrounding area of the assigned position.

9. The patient management system in accordance with claim 1, wherein the patient monitoring device is further configured to trigger a disconnection output if a communication interface is disconnected from one monitored patient identifier of the plurality of patient identifiers.

10. A patient management system in accordance with claim 1, wherein:
role information, comprising data related to an intended role of the user of the corresponding patient identifier from a predefined group of roles, is assigned to a respective patient identifier in the patient monitoring device; and
the predefined group of roles comprises at least one of the following roles: nursing staff member, physician, visitor, trainee, temporary employee.

11. A patient management system in accordance with claim 1, wherein the forwarded signal is processed corresponding to a predefined forwarding instruction.

12. A patient management system in accordance with claim 10, wherein the predefined forwarding instruction pertains to a selection of a subset of roles from the predefined group of roles.

13. A patient management system in accordance with claim 1, wherein the patient identifiers comprise a connection structure configured such that a plurality of patient identifiers can be attached to one another.

14. A patient management system in accordance with claim 1, wherein each of the patient identifiers has a plastic cover or a silicone cover.

15. A patient management system in accordance with claim 1, wherein a respective patient identifier is configured to provide an unambiguous optical identification of the patient assigned to the patient identifier based on one or more of a color, a symbol, a character string, indicia, or mark of the respective patient identifier.

16. A patient management system in accordance with claim 1, further comprising a mobile user carrier device configured with one or more receiving locations, wherein each of the respective patient identifiers is configured to be arranged at the mobile user carrier device of the patient management system.

17. A process for managing an assignment to a patient, the process comprising the steps of:
assigning one of a plurality of patients to a respective patient identifier of a plurality of patient identifiers;
wirelessly outputting the patient-specific information by the respective patient identifier;
detecting and storing an assignment between the patient-specific information and the patient identifier based the output;
receiving a forwarded signal assigned to the patient identifier or to the patient-specific information; and
processing of the forwarded signal based on the stored assignment.

18. A process in accordance with claim 17, further comprising
providing a mobile user carrier device configured with one or more receiving locations, wherein each of the respective patient identifiers is configured to be arranged at the mobile user carrier device of the patient management system; and
arranging a number of patient identifiers at the mobile user carrier device.
